(19) United States
(12) Patent Application Publication
(10) Pub. No.: US 2015/0297398 A1
(43) Pub. Date: Oct. 22, 2015

(54) DISRUPTING ELECTRICAL ACTIVITY IN THE STOMACH

(71) Applicant: MAYO FOUNDATION FOR MEDICAL EDUCATION AND RESEARCH, Rochester, MN (US)

(72) Inventors: Gianrico Farrugia, Rochester, MN (US); Navtej S. Buttar, Rochester, MN (US); Charles J. Bruce, Rochester, MN (US); Samuel J. Asirvatham, Rochester, MN (US); Elizabeth Rajan, Rochester, MN (US); Louis-Michel Wong Kee Song, Rochester, MN (US); Paul A. Friedman, Rochester, MN (US); Juliane Bingener-Casey, Rochester, MN (US); Michael J. Levy, Rochester, MN (US)

(21) Appl. No.: 14/437,913

(22) PCT Filed: Oct. 24, 2013
(86) PCT No.: PCT/US2013/066641

§ 371 Date: Apr. 23, 2015

Related U.S. Application Data
(60) Provisional application No. 61/719,024, filed on Oct. 26, 2012.

(51) Int. Cl. A61F 7/12; A61N 1/36; A61N 1/38; A61N 1/05

(52) U.S. Cl. CPC A61F 7/12; A61N 1/0509; A61N 1/36007; A61N 1/38; A61F 2007/0018

(57) ABSTRACT

This document provides methods and materials involved in disrupting electrical activity in the stomach. For example, methods and materials involved in delivering one or more electrical shocks to the stomach (e.g., the muscularis propria) in a manner that disrupts the normal electrical activity of the stomach (e.g., defibrillating the stomach) are provided.

### CROSS-REFERENCE TO RELATED APPLICATIONS

This application claims the benefit of U.S. Provisional Application Ser. No. 61/719,024, filed Oct. 26, 2012. The disclosure of the prior application is considered part of (and is incorporated by reference in) the disclosure of this application.

### BACKGROUND

1. Technical Field

This document relates to methods and materials involved in disrupting electrical activity in the stomach. For example, this document relates to methods and materials involved in delivering one or more electrical shocks to the stomach (e.g., the muscularis propria) in a manner that disrupts the normal electrical activity of the stomach (e.g., defibrillating the stomach to disrupt the neuro-humeral-interstitial cells of Cajal (ICC)-smooth muscle coordination), thereby reducing caloric intake. This document also relates to methods and materials involved in disrupting the normal electrical activity of the stomach by heating or cooling the stomach to disrupt the neuro-humeral-interstitial cells of Cajal (ICC)-smooth muscle coordination, thereby reducing caloric intake.

2. Background Information

Obesity can be a difficult condition to treat. Treatment methods can include the use of diets, drugs, and physical exercise. Unfortunately, results are usually not long term, and many patients return to their original weight over time. In some cases, invasive approaches such as bypass operations or gastroplasty are used treat obesity. These particular surgical options, however, can be risky and are not appropriate for most patients suffering from obesity.

### SUMMARY

This document provides methods and materials involved in disrupting electrical activity in the stomach. For example, this document provides methods and materials involved in delivering one or more electrical shocks to the stomach (e.g., the muscularis propria) in a manner that disrupts the normal electrical activity of the stomach (e.g., defibrillating the stomach).

As described herein, defibrillating the stomach can disrupt the normal electrical activity of the stomach (e.g., can disrupt the neuro-humeral-ICC-smooth muscle coordination within the stomach) and can result in reduced caloric intake. In some cases, the methods and materials provided herein can be used to induce weight loss and/or to treat obesity. For example, a stomach defibrillator system provided herein can be used to disrupt the normal electrical activity of the stomach in a manner that reduces caloric intake and reduces the body weight of a mammal (e.g., an obese human). In some cases, the methods and materials provided herein can be used treat a metabolic syndrome or a disorder such as anorexia, bulimia, or a motility disorder. In general, one aspect of this document features a method for reducing caloric intake of a mammal. The method comprises, or consists essentially of, delivering defibrillating electrical signals to the muscularis propria of a gastric wall of the mammal under conditions wherein caloric intake of the mammal is reduced. The mammal can be a human. The method can comprise implanting a stomach defibrillator device into the stomach region of the mammal. The stomach defibrillator device can comprise one or more electrodes. The stomach defibrillator device can comprise at least two electrodes. The method can comprise positioning an electrode on either side of the gastric wall. The stomach defibrillator device can comprise a connector. The stomach defibrillator device can comprise an anchor element.

In another aspect, this document features a method for disrupting normal electrical activity of a stomach. The method comprises heating or cooling blood within a gastric vessel. The gastric vessel can be a gastroepiploic artery or vein. The method can comprise implanting a device having a self-contained fluid within the gastric vessel, wherein the device heats or cools the fluid, and wherein the fluid, when heated or cooled, heats or cools blood within the gastric vessel. The method can comprise implanting a device adjacent to the gastric vessel, wherein the device heats or cools blood within the gastric vessel. The device can comprise a coil or cuff that is positioned adjacent to the gastric vessel. The method can comprise implanting a device having a conduit within the gastric vessel, wherein the conduit comprises a heating or cooling element in contact with blood within the gastric vessel, and wherein the heating or cooling element heats or cools blood within the gastric vessel.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF THE DRAWINGS

FIG. 1 is a cross sectional side view of a defibrillator system implanted across a gastric wall of a stomach in accordance with some embodiments provided herein.

FIG. 2 is a cross sectional side view of a defibrillator system implanted within a gastric wall of a stomach (e.g., with the muscularis propria of a stomach) in accordance with some embodiments provided herein.

FIG. 3 is a cross sectional side view of a defibrillator system implanted across a gastric wall of a stomach in accordance with some embodiments provided herein.

FIG. 4 is a cross sectional side view of a defibrillator system implanted across a gastric wall of a stomach in accordance with some embodiments provided herein.

FIG. 5 is a cross sectional side view of a defibrillator system implanted across a gastric wall of a stomach in accordance with some embodiments provided herein.

FIG. 6 is a cross sectional side view of an open system heating/cooling device implanted in a vessel in accordance with some embodiments provided herein.

FIG. 7 is a cross sectional side view of a closed system heating/cooling device implanted in a vessel in accordance with some embodiments provided herein.

FIG. 8 is a cross sectional side view of an extravascular heating/cooling device implanted around a vessel in accordance with some embodiments provided herein.

FIG. 9 is a cross sectional side view of an extravascular heating/cooling device implanted around a vessel in accordance with some embodiments provided herein.

FIG. 10 is a cross sectional side view of a PEG-like defibrillator system implanted across a gastric wall of a stomach in accordance with some embodiments provided herein.

### DETAILED DESCRIPTION

This document provides methods and materials involved in disrupting electrical activity in the stomach. For example, this document provides methods and materials involved in delivering one or more electrical shocks to the stomach (e.g., the muscularis propria) in a manner that disrupts the normal electrical activity of the stomach (e.g., defibrillating the stomach).

As described herein, defibrillating the stomach or disrupting the electrical connectivity between nerves, neuroendocrine cells, interstitial cells of Cajal, and smooth muscle can disrupt the normal electrical activity of the stomach (e.g., can disrupt the neuro-humeral-ICC-smooth muscle coordination within the stomach) and can result in reduced caloric intake. In some cases, the methods and materials provided herein can be used to induce weight loss and/or to treat obesity. For example, a stomach defibrillator system provided herein can be used to disrupt the normal electrical activity of the stomach in a manner that reduces caloric intake and reduces the body weight of a mammal (e.g., an obese human). In some cases, the methods and materials provided herein can be used treat a metabolic syndrome or a disorder such as anorexia, bulimia, or a motility disorder.

The methods and material provided herein can be used to disrupt the normal electrical activity of a stomach of any appropriate mammal and/or can be used to reduce the body weight of any appropriate mammal. For example, the methods and material provided herein can be used to disrupt the normal electrical activity of a human stomach or to reduce the body weight of a human (e.g., a human suffering from obesity).

Any appropriate electrical defibrillator technique can be used to disrupt the normal electrical activity of a stomach, to reduce caloric intake, and/or to reduce the body weight of a mammal. For example, one or more electrodes configured to defibrillate stomach tissue (e.g., the muscularis propria) can be used to disrupt the normal electrical activity of a stomach, to reduce caloric intake, and/or to reduce the body weight of a mammal. In some cases, one or more electrodes configured to defibrillate stomach tissue (e.g., the muscularis propria) as provided herein can be configured to defibrillate stomach tissue without necessarily affecting the level of stomach muscle contraction. For example, one or more electrical shocks can be delivered to stomach tissue in a manner that defibrillates stomach tissue without stimulating the stomach smooth muscle cells. In some cases, one or more electrodes configured to defibrillate stomach tissue (e.g., the muscularis propria) as provided herein can be configured to defibrillate stomach tissue without ablating stomach muscle tissue. For example, one or more electrical shocks can be delivered to stomach tissue in a manner that defibrillates stomach tissue without necessarily ablating the stomach tissue.

In some cases, heating or cooling within the parameters described herein can be used to disrupt the normal electrical activity of a stomach. Electrical activity can be highly sensitive to temperature, and a decrease or increase in temperature can markedly alter electrical activity and propagation of the electrical signal. For example, cooling or heating gastric vessels such as the gastroepiploic artery or vein can alter stomach electrical activity and propagation of the stomach electrical signals. In some cases, cooling or heating can be achieved by cannulating one or more gastric vessels and using a balloon occlusion catheter to vary the temperature of the blood flow with, for example, an implanted device. In some cases, this can be achieved as a periodic outpatient procedure. The implanted device itself can include a pump that circulates saline (e.g., a Peltier or refrigerant pump) and heats or cools the saline (or other fluid in a closed system). The heated or cooled saline can be used to heat or cool the circulated blood. In some cases, an implanted device can heat or cool blood that is simply re-circulated into the vessel (e.g., an open system). In some cases, the implanted device can include a balloon that is used to seat the implanted device in the vessel when the flow and heat exchange is to take place. Such implantable devices can be implanted using any systemic vein or can be implanted via a transhepatic approach to target a gastroepiploic vein.

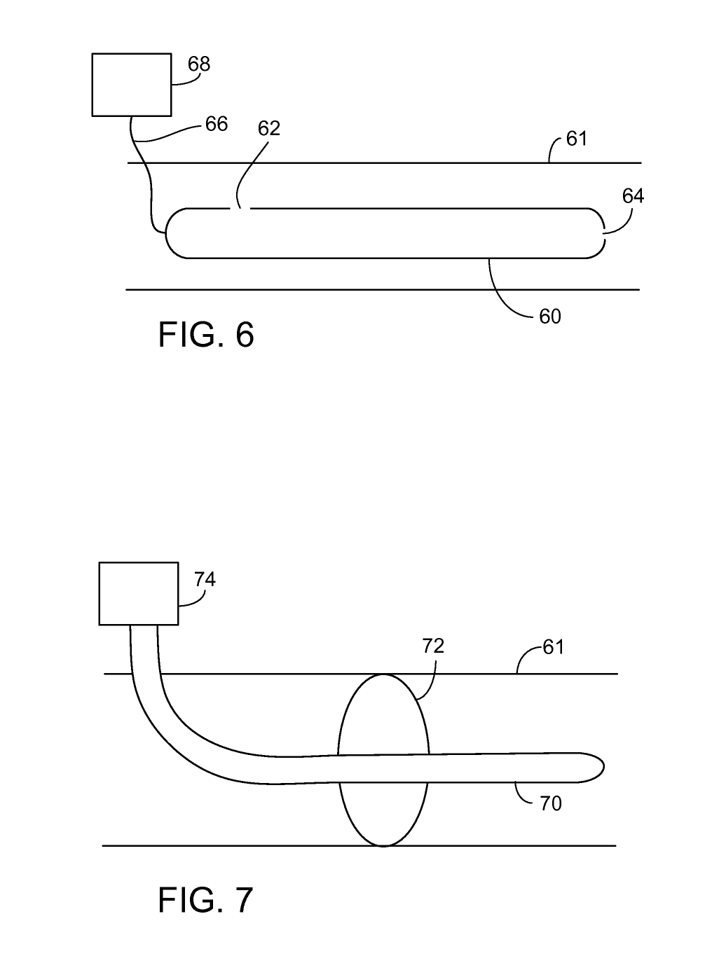

With reference to FIG. 6, an open system implantable device for heating or cooling blood located within vessel 61 can include a temperature conduit 60 attached to a control unit 68 via communication connection 66. Control unit 68 can control the temperature and other treatment parameters (e.g., time) of temperature conduit 60. Temperature conduit 60 can have one or more blood inlets 62 and one or more blood outlets 64. Communication connection 66 can be in the form of a wire or other connection that allows control unit 68 to communicate with temperature conduit 60. Blood entering blood inlet 62 can be heated or cooled to a desired temperature within temperature conduit 60 and can exit via blood outlet 64.

With reference to FIG. 7, a closed system implantable device for heating or cooling blood located within vessel 61 can include a temperature element 70 attached to a control pump 74. Temperature element 70 can be filled with a fluid (e.g., saline). Control pump 68 can control the temperature of the fluid and circulate the fluid (e.g., saline). In some cases, control pump can control other treatment parameters (e.g., time) of the closed system implantable device. In some cases, temperature element 70 can be attached to a balloon 72 configured to slow or restrict the flow of blood to allow the blood to reach a desired temperature. In some cases, a stent or adhesive can be used together with, or in place of, a balloon to maintain the position of the implantable device.

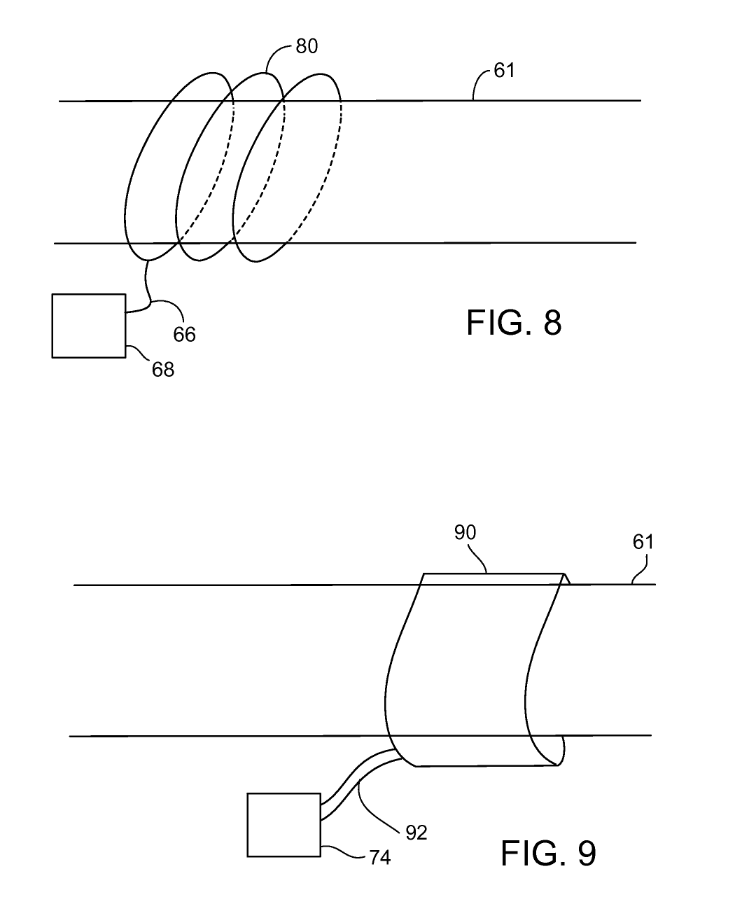

In some cases, heating or cooling within the parameters described herein can be used to disrupt the normal electrical activity of a stomach via an extravascular approach. For example, a coil or cuff can be position adjacent to a vessel and used to heat or cool blood. With reference to FIG. 8, a coil element 80 of an implantable device can be positioned around vessel 61. Coil element 80 can be attached to a control unit 68 via a communication connection 66. With reference to FIG. 9, a cuff element 90 of an implantable device can be positioned around vessel 61. Cuff element 90 can be filled with a fluid or can adjust temperature directly without a fluid. In some cases, cuff element 90 can be attached to a pump or control unit 74 via a connection 92.

The heating or cooling implantable devices can be implanted surgically or endoscopically (e.g., NOTES). A change in temperature of a few degrees (e.g., a change to 33-34° C. for cooling or a change to greater than or equal to 38-42° C. or higher (e.g., up to 100° C.)) can be used.

In some cases, for heating, electrodes can be used and electrode polarity can be changed to decrease clotting and/or to prevent coagulum. For example, phasic heating where polarity is reversed 50-90% of the time can be used. In some cases, the autonomic ganglia and/or peri-cardial autonomic nerves can be targeted with heating, cooling, blocking current, and/or defibrillation. Another example is to cannulate cerebral veins or arteries draining or supplying the lateral and/or ventromedial nuclie of the hypothalamus for defibrillation, stimulation, ablation, and/or periodic cooling.

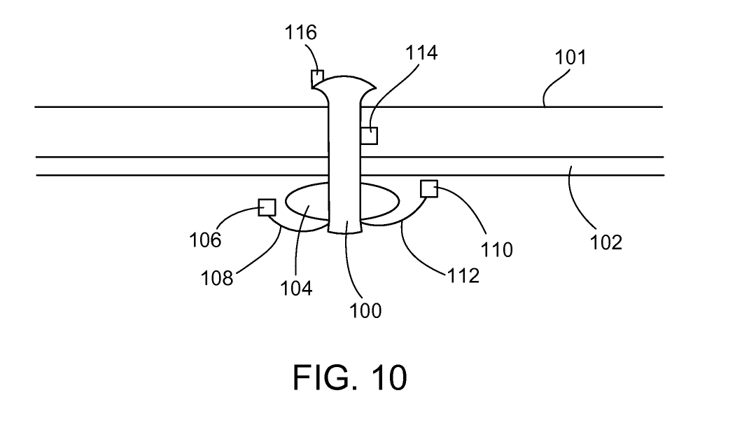

In some cases, a PEG (percutaneous endoscopic gastrostomy)-like approach can be used to place electrodes adjacent to stomach tissue. For example, a tube having one or more electrodes can be introduced into the stomach of a mammal to be treated (e.g., a human). With reference to FIG. 10, a PEG-like tube 100 can be positioned to extend across skin 101 and gastric wall 102 into a stomach. Tube 100 can be hollow and can include a cap on the end positioned outside the patient's body. In some cases, tube 100 can be solid. Tube 100 can include a balloon 104 configured to maintain the position of tube 100. Tube 100 can include a first electrode 106 and one or more second electrodes 110 and 114. In some cases, electrodes 106, 110, and/or 114 can be integral with or incorporated into tube 100 such that they do not extend away from the surface of tube 100. In some cases, electrodes 106, 110, and/or 114 can be integral with or incorporated into balloon 104. In some cases, electrodes 106, 110, and/or 114 extend from tube 100. Electrode 106 can be connected to a power connection port 116 via connection 108. Electrode 110 can be connected to power connection port 116 via connection 112. Electrode 114 can be connected to power connection port 116 via a connection (not shown). Power connection port 116 can be configured to allow for connection to a power supply. In some cases, the patient can ingest conductive fluid to carry current.

Typically, electrical pulses as described elsewhere (e.g., WO02/089655, WO2005/097254, US2002/0165589, or U.S. Pat. No. 6,600,953) are used to stimulate stomach muscle contraction, while electrical pulses as described elsewhere (e.g., US2004/0215180, US2005/0240239, US2010/0145324, or US2005/0096638) are used to ablate stomach tissue. Electrical shocks designed to defibrillate stomach tissue can be monophasic, biphasic, or custom shock waveforms (e.g., customized based on timing and relative contribution of both phases of the shock) at 1 joule to 200 joule range (e.g., 1 joule to 150 joule, 1 joule to 100 joule, 10 joule to 200 joule, 50 joule to 200 joule, or 50 joule to 150 joule), monopolar or bipolar. In each of these iterations, the reference electrode or bipole can be placed endoluminally, subserosal, or have the patches and electrodes external to the body (e.g., on the skin or capacitively linked). The timing of the phases, as well as energy delivery, can be timed to avoid the cardiac vulnerable period (synchronized) or if bipolar, done in an asynchronous manner. In some cases, the stomach contractions can be timed, and shocks delivered sequentially with multiple electrodes in a linear, annular, or spiral format optionally linked with stomach contractility and anatomic location. These parameters are not the same as typical stimulatory trains for pacing and do not involve radiofrequency or cryo ablative energy delivery used in stomach stimulation and stomach tissue ablation.

In some cases, a device provided herein configured to defibrillate stomach tissue also can be configured to provide electrical stimulation signals and/or tissue ablation to stomach tissue. For example, a stomach defibrillator system provided herein can be configured to deliver electrical stimulation signals and/or tissue ablation to stomach tissue in addition to delivering one or more electrical shocks configured to defibrillate stomach tissue. Examples of electrical stimulation signals that can be delivered using a stomach defibrillator system provided herein include, without limitation, those electrical stimulation signals described elsewhere (e.g., WO02/089655, WO2005/097254, US2002/0165589, or U.S. Pat. No. 6,600,953). Examples of electrical ablation signals that can be delivered using a stomach defibrillator system provided herein include, without limitation, those electrical ablation signals described elsewhere (e.g., US2004/0215180, US2005/0240239, US2010/0145324, or US2005/0096638). In some cases, a device provided herein configured to defibrillate stomach tissue also can be configured to provide mechanical injury to stomach tissue.

In some cases, a stomach defibrillator system provided herein can be implanted (e.g., implanted endoscopically or laparoscopically) into a mammal (e.g., a human) such that one or more electrodes of the stomach defibrillator system are positioned within a gastric wall of a stomach (e.g., within the muscularis propria) or proximal to a mucosal surface or a serosal surface of a gastric wall of a stomach. For example, electrodes configured to defibrillate stomach tissue can be positioned less than a millimeter from a serosal surface of a gastric wall of a stomach to defibrillate stomach tissue. In some cases, a stomach defibrillator device provided herein can include one or more (e.g., one, two, three, four, five, or more transmural anchor components configure to hold the stomach defibrillator device within a particular position. In some cases, the electrodes of a stomach defibrillator device provided herein can be small electrodes on a gastric fundus or a gastric body or antrum (e.g., on a larger fundic cap). In some cases, a battery powered control unit of a stomach defibrillator device can be implanted (e.g., in the patient's abdomen or subcutaneously) and can have one or more extensions connecting the control unit to one or more electrodes positioned at a targeted stomach locations (e.g., muscularis propria). In some cases, the control unit can wirelessly communicate with a stomach defibrillator device.

Any appropriate defibrillating electrical signals can be used provided that they defibrillate stomach tissue. For some applications, defibrillating electrical signals can be used only during eating periods or periods following eating periods (e.g., for one to two hours following eating a meal). In some cases, a stomach defibrillator device provided herein can be configured to deliver direct current or other types of energy (e.g., non-thermal radio frequencies at about, for example, 30 Hz, mechanical vibrations, phototherapy, light, and the like). In some cases, a stomach defibrillator device provided herein can be configured to deliver a direct current offset (e.g., a constant direct current offset) to decrease the infection rate of the implant. In some cases, a stomach defibrillator device provided herein can be configured in a bipolar or monopolar manner. For example, a stomach defibrillator device provided herein can be configured to deliver defibrillating electrical signals in a mucosa to serosa, a mucosa to muscularis propria, or a mucosa to mucosa manner. Such local signals can be delivered in a bipolar manner (e.g., serosa to mucosa) to minimize stimulation of extra-gastric tissues.

In some cases, defibrillating electrical signals can have waveforms that are monophasic, bipolar, capacitor discharges. In some cases, stimulation with lower energy can be accomplished using an ascending ramp or modified ascending ramp waveform.

In some cases, defibrillating electrical signals can be used to defibrillate stomach tissue for certain periods of a day. For example, defibrillating electrical signals can be used to defibrillate stomach tissue during the night and not during the day, or can be used to defibrillate stomach tissue during the day and not during the night. In some cases, a stomach defibrillator device provided herein can be configured to include one or more sensors configured to detect movement, meal intake, the time of day, ablation effectiveness when the stomach defibrillator device is configured to ablate tissue, temperature, impedance, or pressure. In such cases, a stomach defibrillator device provided herein can deliver the desired defibrillating electrical signals based on inputs from the one or more sensors. For example, a change in pH associated with entry of food into the stomach or small bowel can be used to trigger defibrillation. In some cases, a stomach defibrillator device provided herein can be configured to include the ability to collect local electograms. For example, a stomach defibrillator device provided herein can be configured to include one or more electrodes to collect local electograms.

In some cases, a gastric defibrillator can be designed with one or more filters to permit sensing of cardiac signals. Such filters can allow synchronization of gastric electrical therapies to cardiac electrical activity. Delivery of larger energy gastric defibrillation can be timed to the cardiac QRS to minimize the risk of cardiac pro-arrhythmia. In some cases, a patient control unit can be used to allow patient modulation of device activity and/or outputs and sensing functions. For example, if there was discomfort with the therapy, outputs can be reduced, or in the absence of effectiveness, outputs can be increased, or activity activated around mealtimes.

In some cases, one or more than one location of a mammal's stomach can be target to defibrillate stomach tissue. For example, two or more stomach defibrillator devices can be implanted into a mammal to defibrillate stomach tissue. In some cases, one or more electrodes of a stomach defibrillator device provided herein can be configured to avoid an edge effect. For example, edge effects can be avoided by having rounded margin electrodes, irrigation ports that use either stomach contents or external irrigation, phasic energy delivery to longer linear electrodes, and/or sequential defibrillation. Avoidance of edge effects can allow for repetitive use from the same electrode position for the iterations of this device that involved triggering with meals or painless defibrillation at predetermined intervals.

In some cases, a stomach defibrillator device provided herein can be configured to include one or more protective sinks (e.g., one, two, three, four, five, six, or more protective sinks) to avoid delivering signals to other tissues (e.g., pylorus tissue). For example, a stomach defibrillator device provided herein can be configured to avoid delivering defibrillating electrical signals to pylorus tissue. In some cases, a stomach defibrillator device provided herein can be configured to elute gel, fluid, or an adhesive. For example, unwanted collateral defibrillation can be minimized using designs where the defibrillatory energy is sent between two electrodes, both at the target site for delivery (local, bipolar defibrillation). In some cases, insulation and shielding to channel a defibrillation vector when non-bipolar systems are used can be accomplished by using an insulatory fabric or nonconductive gel on the edges of the defibrillator patch and on the portion not directly in contact with stomach tissue.

Any appropriate method can be used to implant a stomach defibrillator device provided herein. For example, initial access can be carried out under endoscopic ultrasound with a 19- to 22-gauge needle. In some cases, a needle and/or guidewire can be used with or without electrodes. For example, a needle containing electrodes can be used to place electrodes within the muscularis propria or across the stomach wall. In order to have local tissue effects of defibrillation and to minimize collateral defibrillatory energy delivery, the electrodes can be placed within the stomach tissue rather than a bipole system used where the electrodes are placed on either side (endoluminal and serosal) of the stomach tissue. This type of electrode configuration can facilitate sequential defibrillatory energy delivery, which can facilitate effects particularly when timed with the stomach wavefront of activation variant. In some cases, an ablation catheter optionally with irrigation and/or suction ports can be used. In such cases, a deflectable catheter can be placed at a determined site (e.g., a site determined either by mapping or empiric anatomic localization) for enhanced density of energy delivery. This can serve as a return electrode for the defibrillation vector. This catheter can be manipulated in the vasculature, and in that instance, irrigation can be used both to increase the surface area of the electrode and to minimize coagulum formation. In some cases, a separate ultrasound probe can be placed in the stomach to track a placement catheter after initial needle placement. In some cases, a contrast agent can be injected into the vasculature or into the stomach and used to position a stomach defibrillator device provided herein via fluoroscopic guidance. Any appropriate contrast agent (e.g., iodinated contrast either ionic or non-ionic) can be used in the vasculature or as an oral contrast agent. For example, gastrografin can be used for assessing luminal placement.

In some cases, a stomach defibrillator device provided herein can include a defibrillation patch that is positioned within the muscularis propria with a second patch either in the gastric lumen or punched out. For example, the second patch can be punched out externally like a t-tag through the gastric wall.

In some cases, a device provided herein can be configured to deliver vibration in place of or in addition to defibrillating electrical signals. The use of vibration can leave receptors intact and can affect contractility. In some cases, vibration can be used at a level that is below an ultrasound level as a standalone energy source with its mechanical effects with no acoustic or ultrasound effects. In some cases, a device provided herein that is configured to deliver vibration can include one or more joints configured to create vibrations.

In some cases, venous ablation can be performed to treat obesity. For example, an electrode (e.g., a linear electrode) can be placed in a gastric wall vein for ablation (e.g., bipolar ablation) of the targeted vessel to treat obesity.

In some cases, electrodes, cooling capsules, and/or heating capsules can be swallowed. The electrodes themselves may be metallic, conductive, or liquid (hypertonic saline as a virtual electrode). The return electrode can be permanently placed in the stomach wall serosally or within the stomach muscle itself. Energy can be delivered after the electrodes have been swallowed so as to minimize collateral effects and allow increased energy delivery.

The methods and materials described herein can be used to target sites in addition to the stomach or instead of the stomach. For example, the methods and materials described herein can be used treat the small bowel, bladder, hypothalamus (e.g., hypothalamus cooling), vagal nerve (e.g., cooling and optionally stimulation), and/or celiac ganglia (e.g., cooling or RF). In some cases, a device provided herein can be implanted near the anus to control incontinence.

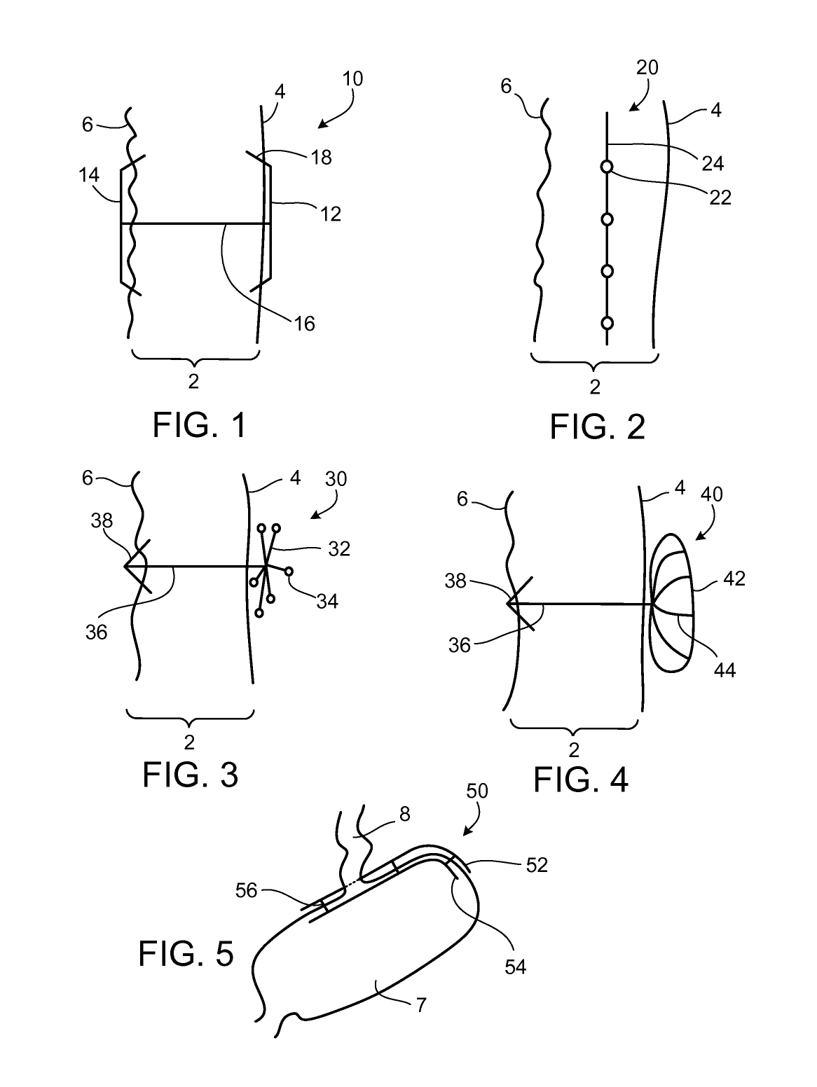

With reference to FIG. 1, a stomach defibrillator device 10 can include an electrode 12 and an electrode 14 connected via a connector 16. Electrodes 12 and 14 can be positioned on either side of gastric wall 2 of a mammal (e.g. a human). For example, electrode 12 can be proximal to a serosal surface 4, and electrode 14 can be proximal to a mucosal surface 6. In some cases, electrodes 12 and 14 can include anchor elements 18 configured to position electrodes 12 and 14 within a particular location with minimal or no movement.

With reference to FIG. 2, a stomach defibrillator device 20 can include one or more electrodes 22 connected via connectors 24. Electrodes 22 can be positioned within gastric wall 2 of a mammal (e.g. a human). For example, electrodes 22 can be positioned within the muscularis propria of gastric wall 2.

With reference to FIG. 3, a stomach defibrillator device 30 can include one or more electrodes 34. Electrodes 34 can be interconnected via connectors 32 and can be anchored to a gastric wall 2 of a mammal (e.g. a human) via a stem 36 and anchor elements 38. Stem 36 and anchor elements 38 can be configured to position stomach defibrillator device 30 in a particular location with minimal or no movement. Electrodes 34 can be positioned on either side of gastric wall 2. For example, electrodes 34 can be proximal to a serosal surface 4.

With reference to FIG. 4, a stomach defibrillator device 40 can include one or more electrodes 44. Electrodes 44 can be located on a balloon 42 (e.g. an inflatable balloon). Balloon 42 can be attached to a stem 36 and anchor elements 38. Stem 36 and anchor elements 38 can be configured to position stomach defibrillator device 40 in a particular location with minimal or no movement. Electrodes 44 and balloon 42 can be positioned on either side of gastric wall 2. For example, electrodes 44 and balloon 42 can be proximal to a serosal surface 4.

With reference to FIG. 5, an electrode 52 and an electrode 54 connected via a connector 56. Electrodes 52 and 54 can be positioned on either side of a gastric wall of a mammal (e.g. a human). For example, electrode 52 can be proximal to a serosal surface, and electrode 54 can be proximal to a mucosal surface. In some cases, electrodes 52 and 54 can be positioned to provide electrical signals to a large portion of a stomach 7 around the area where an esophagus 8 attached to stomach 7.

The invention will be further described in the following examples, which do not limit the scope of the invention described in the claims.

### EXAMPLES

### Example 1

### Determining the Effects of Acute Stomach Defibrillation

Acute group guinea pigs were subjected to an in vivo procedure to assess and calibrate the effects of defibrillation. Briefly, guinea pigs were anesthetized with isoflurane for the purpose of placement of external electrodes and defibrillation of the stomach region. An incision was made to gain access to the stomach and electrodes placed on the gastric serosa to record electrical activity. The effects of external defibrillation were examined to determine optimal energy delivery parameters. External defibrillation was conducted using 1, 2, 3, 5, 10, 20, and 50 joules without inducing ventricular fibrillation. There was no injury to the abdominal wall. An examination of the stomach after defibrillation revealed a decrease in contractile activity.

### Example 2

### Determining the Effects of Chronic Stomach Defibrillation

Guinea pigs are anesthetized with isoflurane for the purpose of placement of external electrodes and defibrillation. A sham group is anesthetized with no defibrillation. In another control, guinea pigs are anesthetized, and the left leg is defibrillated using the same energy applied to the abdomen. The energy delivered to the abdomen is 10 joules. After the treatment is completed, guinea pigs are left unrestrained and are returned to the animal facility immediately. Daily weight measurements and food intake monitoring are conducted for 10 days. After 10 days, the guinea pigs are terminated. The sample size for these experiments is 6.

In another procedure, guinea pigs are anesthetized with isoflurane for the purpose of placement of external electrodes and defibrillation. The sham group is anesthetized with no defibrillation. In another control, guinea pigs are anesthetized, and the left leg is defibrillated using the same energy applied to the abdomen. The energy delivered to the abdomen is 10 joules. After the treatment is completed, guinea pigs are left unrestrained and are returned to the animal facility immediately. Treatment is repeated each day for 5 consecutive days. Daily weight measurements and food intake monitoring are conducted during the treatment period and for 5 additional days. After 10 days, the guinea pigs are terminated. The sample size for these experiments is 6.

In each case, anesthesia is induced via a fume hood. To deliver defibrillation, one electrode is placed on the abdomen, and one is placed on the hindquarters or back to serve as a grounding pad or electrode. Defibrillation is delivered as a pulse. The pulse duration is less than one second, which is delivered automatically via the defibrillation equipment.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method for reducing caloric intake of a mammal, wherein said method comprises delivering defibrillating electrical signals to the muscularis propria of a gastric wall of said mammal under conditions wherein caloric intake of said mammal is reduced.

2. The method of claim 1, wherein said mammal is a human.

3. The method of claim 1, wherein said method comprises implanting a stomach defibrillator device into the stomach region of said mammal.

4. The method of claim 3, wherein said stomach defibrillator device comprises one or more electrodes.

5. The method of claim 3, wherein said stomach defibrillator device comprises at least two electrodes.

6. The method of claim 5, wherein said method comprises positioning an electrode on either side of said gastric wall.

7. The method of claim 3, wherein said stomach defibrillator device comprises a connector.

8. The method of claim 3, wherein said stomach defibrillator device comprises an anchor element.

9. A method for disrupting normal electrical activity of a stomach, wherein said method comprises heating or cooling blood within a gastric vessel.

10. The method of claim 9, wherein said gastric vessel is a gastroepiploic artery or vein.

11. The method of claim 9, wherein said method comprises implanting a device having a self-contained fluid within said gastric vessel, wherein said device heats or cools said fluid, and wherein said fluid, when heated or cooled, heats or cools blood within said gastric vessel.

12. The method of claim 9, wherein said method comprises implanting a device adjacent to said gastric vessel, wherein said device heats or cools blood within said gastric vessel.

13. The method of claim 12, wherein said device comprises a coil or cuff that is positioned adjacent to said gastric vessel.

14. The method of claim 9, wherein said method comprises implanting a device having a conduit within said gastric vessel, wherein said conduit comprises a heating or cooling element in contact with blood within said gastric vessel, and wherein said heating or cooling element heats or cools blood within said gastric vessel.
